(19) United States
(12) Patent Application Publication
(10) Pub. No.: US 2018/0228758 A1
(43) Pub. Date: Aug. 16, 2018

(54) COMPOSITIONS AND METHODS TO TREAT INFECTED EAR CONDITIONS

(71) Applicant: Hennepin Life Sciences, LLC, Plymouth, MN (US)

(72) Inventors: Patrick M. Schlievert, Iowa City, IA (US); Marnie L. Peterson, Jackson, WY (US)

(21) Appl. No.: 15/753,002

(22) PCT Filed: Aug. 9, 2016
(86) PCT No.: PCT/US2016/046161

§ 371 Date: Feb. 15, 2018

Related U.S. Application Data
(60) Provisional application No. 62/206,951, filed on Aug. 19, 2015.

(51) Int. Cl. A61K 31/23; A61K 45/06; A61K 9/06; A61K 47/44; A61K 47/38; A61P 31/04

(52) U.S. Cl. CPC A61K 31/23; A61K 45/06; A61P 31/04; A61K 47/44; A61K 47/38; A61K 9/06

(57) ABSTRACT

Methods of treating Otitis externa by inhibiting the growth of, or killing, certain microorganisms associated with this condition are disclosed. Prophylaxis methods of inhibiting Otitis externa infections are also disclosed.

### PRIORITY

This application claims the benefit of U.S. Provisional Application No. 62/206,951, entitled “COMPOSITIONS AND METHODS TO TREAT INFECTED EAR CONDITIONS” and filed Aug. 19, 2015, the entirety of which is incorporated herein by reference.

### BACKGROUND

This invention relates to controlling undesired infected or infested ear conditions, and more particularly to controlling these ear conditions in animals.

Dogs and cats are susceptible to a variety of undesired conditions and infections of the external ear canals. The condition may be caused by ear mite (arachnid) infections, often caused by, but not limited to Otodectes cynotis. This infection is very highly contagious to other animals. Symptoms of ear mite infection include the following: excessive scratching and rubbing of ears, head shaking, black or brown waxy secretion, strong odor, inflammation, and obstruction of ear canal with coffee ground-like discharge. These infections may also predispose the animal to additional bacterial and yeast infections.

Dogs with floppy ears and those that enjoy swimming often acquire other conditions, such as bacterial and yeast infections, characterized by similar symptoms as ear mite infections. Typically, there is a very strong odor due to the causative microbes. Common bacterial causes include the gram-positive Staphylococcus aureus and gram-negative Pseudomonas species and Proteus vulgaris or mirabilis. An example of a common yeast infection is candidiasis.

### SUMMARY

The invention provides a method of treatment or prophylaxis for Otitis externa in animals with a gel-based composition. The gel-based composition includes one or more compounds (e.g., glycerol monolaurate, (GML)) that (i) kill or inhibit the growth of the infectious pests and microorganisms. In some embodiments, the gel-based composition is a compound of either Formula I or Formula II, or both Formula I and Formula II.

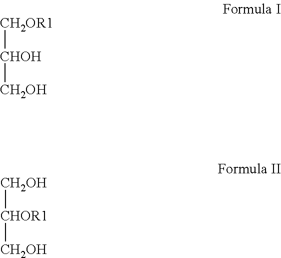

Wherein R1 may be CO(CH₂)₁₀CH₃.

In an exemplary embodiment, the gel-based composition includes GML. GML may be present in an amount of about 10-100 mg/mL, or about 30-70 mg/mL. The composition may also, or alternatively, include glycerol, vegetable oil, and/or petroleum jelly. In further embodiments, the composition may further include vegetable oil, a cellulose derivative, one or more accelerants, an additional active ingredient, or combinations thereof. A suitable additional active ingredient may be selected from an anti-biotic, anti-viral, anti-fungal, or a combination thereof.

Another embodiment is a method of treatment or prophylaxis comprising identifying the subject likely to have been exposed to an infectious microorganism or mite. The microorganism or mite may be selected from Otodectes, Staphylococcus, Pseudomonas, or Proteus. The infected subject is administered a gel-based composition that kills or inhibits the growth of the infections microorganism or mite and the gel-based composition comprises a compound of either Formula I or Formula II, or both Formula I and II (illustrated above).

The gel may be one or more of: glycerol, vegetable oil, propylene glycol, petroleum jelly, or other similar compounds.

In an alternative embodiment, the gel based composition contains compounds that are either Formula III or Formula IV, or both Formula III and Formula IV.

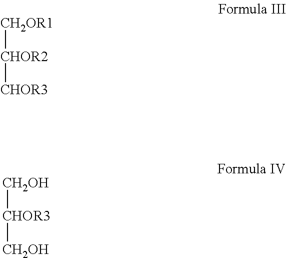

wherein R1 may be: H, CO(CH₂)₈CH₃, CO(CH₂)₁₀CH₃, or CO(CH₂)₁₂CH₃;

R2 may be: H, CO(CH₂)₈CH₃, CO(CH₂)₁₀CH₃, CO(CH₂)₁₂CH₃, O(CH₂)₉CH₃, O(CH₂)₁₁CH₃, or O(CH₂)₁₃CH₃, and

R3 may be: CO(CH₂)₈CH₃, CO(CH₂)₁₀CH₃, CO(CH₂)₁₂CH₃, O(CH₂)₉CH₃, O(CH₂)₁₁CH₃, or O(CH₂)₁₃CH₃.

The gel-based compositions can be administered either before, simultaneous with, or after the administration of one or more supplementary agents. Supplementary agents can include, for example, anti-fungal agents, modulators of immune function, or antibiotics.

Compositions containing one or more pharmaceutical excipients and one or more gel-based compositions or one or more gel-based compositions alone can also be included in gels, creams, and foams.

### BRIEF DESCRIPTION OF THE DRAWINGS

FIG. 1 is a graph showing the effects of various concentrations of GML in non-aqueous gels on the growth of Proteus species.

FIG. 2 is a series of graphs showing the measured CFU/mL from biofilms formed from S. aureus strains MN8 (methicillin sensitive strain, top panels), MNWH (methicillin resistant strain, middle panels), and MW2 (methicillin resistant strain, bottom panels) cultured in 96 well plastic microtiter plates, in the presence of 500 μg/mL GML for 24 or 48 hours.

FIG. 3 is a series of graphs showing the measured biofilm absorbance at 595 nm after crystal violet staining of biofilms, formed from S. aureus strains MN8 (methicillin sensitive strain, top panels), MNWH (methicillin resistant strain, middle panels), and MW2 (methicillin resistant strain, bottom panels) cultured in 96 well plastic microtiter plates, for 24 or 48 hours, in the presence or absence of the indicated concentration of GML in a non-aqueous gel.

FIG. 4 is a graph showing CFU/mL from Pseudomonas aeruginosa cultures grown at the indicated pH and at the indicated concentrations of GML in a non-aqueous gel.

### DETAILED DESCRIPTION

The present invention provides topical GML compositions and methods of treatment with the compositions, e.g., by topical administration. The disclosed GML compositions and methods, in one embodiment, are used for treating infections topically, for example, ear infections by delivery of effective amounts of GML, or a derivative thereof, to a skin or mucosal surface of a subject, e.g., a dog, cat, horse, or rabbit.

“Antimicrobial,” as used herein, means effective in preventing, inhibiting, or arresting the growth or pathogenic effects of a microorganism. “Microorganism” as used herein means any bacteria, virus, or fungus. In one embodiment, the compositions of the invention are used to prevent, inhibit, or arrest the growth of one or more of the following microorganisms: Otodectes cynotis, Staphylococcus aureus, Pseudomonas aeruginosa, and Proteus vulgaris or mirabilis.

“Anti-bacterial” or “anti-fungal,” as used herein refers to inhibition or arrest of the growth of a bacterium or fungus, a reduction in the severity of or likelihood of developing a bacterial or fungal disease, inducing death of the bacterium or fungus or reduction or inhibition of the pathogenic effects of the respective bacterium or fungus. “Bactericidal” is used interchangeably with “anti-bacterial.”

“Anti-viral,” as used herein, refers to inhibition of viral infection or virus replication, a reduction in the likelihood that a subject exposed to a virus will contract the viral disease or a reduction in the severity of the viral disease.

“Effective amount,” as used herein, refers to an amount that is sufficient to affect a beneficial or desired antimicrobial activity, including, without limitation, killing the microorganism or inhibiting microbial infection, growth or toxicity. An effective amount of GML is about up to 10 μg/mL, about up to 100 μg/mL, about up to 1 mg/mL, about up to 10 mg/mL, about up to 50 mg/mL, or about up to 100 mg/mL.

“Treat,” “treatment,” and “treating,” as used herein, refer to an approach for obtaining beneficial or desired results, for example, clinical results. For the purposes of this invention, beneficial or desired results may include inhibiting or suppressing the growth of a microorganism or killing a microorganism; inhibiting one or more processes through which a microorganism infects a cell or subject; inhibiting or ameliorating the disease or condition caused by a microbial infection; inhibiting or killing a mite, insect, arachnoid, or spider, or a combination thereof.

The terms “treat,” “treatment,” or “treating” also refer to prophylaxis treatment. “Prophylaxis,” as used herein, can mean complete prevention of an infection or disease, or prevention of the development of symptoms of that infection or disease; a delay in the onset of an infection or disease or its symptoms; or a decrease in the severity of a subsequently developed infection or disease or its symptoms.

“Subject,” as used herein, includes animals, such as domestic animals. The subject, in some embodiments, is a dog, cat, or horse.

“Topical,” as used herein, refers to the application of the composition to any skin or mucosal surface. “Skin surface” refers to the protective outer covering of the body of a vertebrate, generally comprising a layer of epidermal cells and a layer of dermal cells. “Mucosal surface,” refers to a tissue lining of an organ or body cavity that secretes mucous.

“Pharmaceutically acceptable topical carrier,” as used herein, refers to a material, diluent, or vehicle that can be applied to skin or mucosal surfaces without undue toxicity, irritation, or allergic reaction.

“Pharmaceutically acceptable excipient,” as used herein, means an excipient that is useful in preparing a pharmaceutical composition that is generally safe, non-toxic and neither biologically nor otherwise undesirable, and includes an excipient that is acceptable for veterinary use as well as human pharmaceutical use. A pharmaceutically acceptable excipient includes both one and more than one such excipient.

“Vegetable oil,” as used herein, means a substance extracted from a plant or seed that exists in liquid form at room temperature. Suitable vegetable oils include, without limitation, palm, olive, corn, canola, coconut, soybean, wheat germ, jojoba, sunflower, sesame, peanut, cottonseed, safflower, soybean, rapeseed, almond, beech nut, cashew, hazelnut, macadamia, mongongo nut, pecan, pine nut, pistachio, walnut, grapefruit seed, lemon, orange, bitter gourd, bottle gourd, buffalo gourd, butternut squash seed, egusi seed, pumpkin seed, watermelon seed, acai, black seed, blackcurrant seed, borange seed, evening primrose, flaxseed, eucalyptus, amaranth, apricot, apple seed, argan, avocado, babassu, coriander seed, grape seed, mustard, poppyseed, rice bran, castor, or mixtures thereof. Mixtures can be, for example, a combination of olive oil and soybean oil, a combination of coconut oil and wheat germ oil, or a combination of jojoba oil, palm oil, and castor oil. Mixtures of vegetable oils can be binary, ternary, quaternary, or higher mixtures.

“Accelerant,” as used herein, refers to a compound, substance, liquid, powder, or mixture that, when added to the composition, has the effect of enhancing or contributing to the antimicrobial properties of the composition. In some embodiments, the accelerant is selected from an antibiotic agent, anti-fungal agent, anti-viral agent, or combination thereof. Antibiotics include, for example, aminoglycosides, carbacephems, cephalosporins, glycopeptides, lincosamides, lipopetides, macrolides, monobactams, nitrofurans, penicillins, polypetides, quinolones, sulfuramides, or tetracyclines. Anti-fungal agents include, for example, those of the azole class, polyene class, or echinocanins class, nucleoside analogues, allylamines, griseofulvin, tolnaftate, or selenium compounds. Anti-viral agents include, for example, acyclovir, ganciclovir, valganciclovir, abacavir, enofovir, lamivudine, emtricitabine, zidovudine, tenofovir, efavirenz, raltegravir, enfuvirdide, maraviroc, ribavirin, amantadine, rimantadine, interferon, oseltamivir, or zanamivir.

“Cellulose derivative,” as used herein, refers to any a cellulose-based compound and may include, for example, hydroxyethyl cellulose, hydroxypropyl cellulose, methylcellulose, ethyl cellulose, hydroxypropyl methyl cellulose, or cellulose acetate.

“Biofilm,” as used herein, means an aggregate of microorganisms, usually bacterial, adhered to one another and growing on a surface. The microbial cells in the biofilm typically produce an extracellular matrix known as an extracellular polymeric substance. Often, this matrix and the density of the aggregate itself significantly increase the antibiotic resistance of the bacteria in the biofilm. Biofilms can be involved in ear infections and dental diseases such as gingivitis.

“Isolated compound”, as used herein refers GML or a related compound that either has no naturally-occurring counterpart or has been separated or purified from components which naturally accompany it, e.g., in tissues such as pancreas, liver, spleen, ovary, testis, muscle, joint tissue, neural tissue, gastrointestinal tissue or tumor tissue, or body fluids such as blood, serum, or urine. Typically, a naturally occurring biological compound is considered “isolated” when it is at least 70%, by dry weight, free from other naturally-occurring organic molecules with which it is naturally associated. Preferably, a preparation of a compound for use in the invention is at least 80%, more preferably at least 90%, and most preferably at least 99%, by dry weight, that compound. The degree of isolation or purity can be measured by any appropriate method, e.g., column chromatography, polyacrylamide gel electrophoresis, or HPLC analysis. Since a compound (e.g., GML) that is chemically synthesized is, by its nature, separated from the components that naturally accompany it, the synthetic compound is by definition “isolated.”

Isolated compounds, and supplementary agents useful for the invention, can be obtained, for example, by: (i) extraction from a natural source (e.g., from tissues or bodily fluids); (ii) where the compound or supplementary agents are proteins, by expression of recombinant nucleic acids encoding the proteins; or (iii) by standard chemical synthetic methods known to those in the art.

In one embodiment, the present invention provides a topical composition comprising GML or a derivative thereof. In another embodiment, the composition comprises a vegetable oil or a non-aqueous gel, or a combination thereof. In one embodiment, the non-aqueous gel comprises a cellulose derivative. In one embodiment, the topical composition comprises a pharmaceutically acceptable topical carrier.

GML is a fatty acid ester of glycerol, derivative of lauric acid, with the chemical formula C₁₅H₃₀O₄. GML is also known in the art as glyceryl laurate or monolaurin. GML is found naturally in breast milk and some plants, and is used as a food and cosmetic additive. GML and other glycerides are listed in the Generally Recognized as Safe Substances database by the US Food and Drug Administration. GML and related compounds have been previously disclosed, for example, in U.S. patent Publication No. 2007/0276049 (filed Nov. 10, 2004) and U.S. Pat. No. 8,796,332, the disclosures of which are herein incorporated by reference for all purposes.

GML can be obtained or synthesized in multiple forms including both R and S optical isomers, as well as forms with lauric acid in the ⅓-position and in the 2-position. The disclosed composition, in one embodiment, comprises the R isomer of GML. In another embodiment, the disclosed composition comprises the S isomer of GML. In yet another embodiment, the composition comprises a racemic mixture of R and S isomers.

Similarly, the topical composition may comprise GML with lauric acid ester at the ⅓ position, GML with lauric acid ester at the 2-position, or a combination thereof. R and S isomers of each form and racemic mixtures are suitable for use with the present invention.

The structure is glycerol monolaurate (GML) ⅓-position (lauric acid in the 1 or 3 position) is:

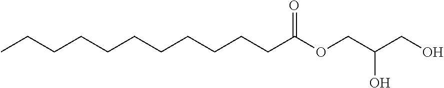 [SMILES: CCCCCCCCCCCC(=O)OCC(O)CO]

The structure of glycerol monolaurate (GML) 2-position is:

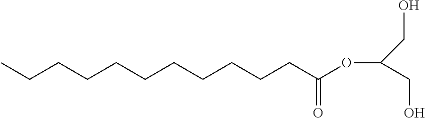 [SMILES: CCCCCCCCCCCC(=O)OC(CO)CO]

In another embodiment, the topical composition comprises a GML derivative, for example a compound selected from one of Formulas A-F. Examples of such compounds include, for example, glycerol monocaprylate, glycerol monocaprate, glycerol monomyristate, glycerol monopalmitate, and dodecyl glycerol.

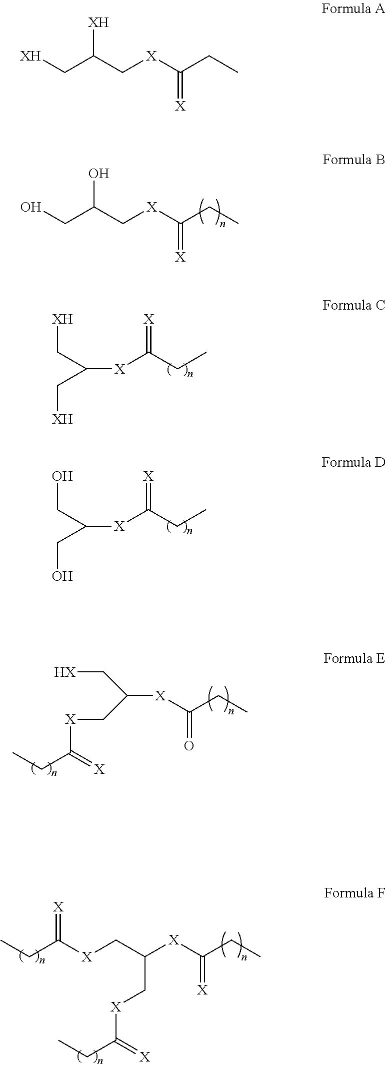

wherein each occurrence of X is independently —O— or —S—; and n is an integer from 5 to 20 (inclusive).

In another embodiment, the topical composition comprises at least one derivative of GML, and the at least one derivative is a compound of either Formula E or Formula F. Examples of such compounds include, for example, glycerol dilaurate, glycerol dicaprylate, glycerol dimyristate, glycerol trilaurate, and glycerol tripalmitate.

In one embodiment, a compound of Formula A, B, C, or D is present in the topical composition of the invention, and at least one —X— is —S—. In one embodiment, one occurrence of —X— is —S— and the remaining occurrences of —X— are —O—.

In one embodiment, a compound of Formula E or F is present in the topical composition of the invention, each occurrence of n is 10, and at least one —X— is —O—. In one embodiment, the topical composition comprises GML and a compound of Formula F. In a further embodiment, each occurrence of n is 10 and at least one —X— is —O—.

In another embodiment, the topical composition comprises GML or derivative thereof at a concentration of about 10 μg/mL to about 100 mg/mL. In a further embodiment, the topical composition comprises GML or derivative thereof at a concentration of about 50 μg/mL to about 50 mg/mL. In a further embodiment, the topical composition comprises GML or derivative thereof at a concentration of about 100 μg/mL to about 10 mg/mL. In yet a further embodiment, the topical composition comprises GML or a derivative thereof at a concentration of about 500 μg/mL to about 5 mg/mL.

In one embodiment, the topical composition comprises GML or derivative thereof at a concentration of about 10 μg/mL, about 50 μg/mL, about 100 μg/mL, about 500 μg/mL, about 1 mg/mL, about 5 mg/mL, about 10 mg/mL, about 50 mg/mL, or about 100 mg/mL.

Exemplary GML Concentrations

| | | |
|---|---|---|
| 0.001% | 10 | μg/mL |
| 0.01% | 100 | μg/mL |
| 0.1% | 1 | mg/mL |
| 1% | 10 | mg/mL |
| 2.5% | 25 | mg/mL |
| 5% | 50 | mg/mL |
| 7.5% | 75 | mg/mL |
| 10% | 100 | mg/mL |

The amount of GML or derivative thereof in the composition may be tailored to the extent of the ear infection being treated as well as to the characteristics of the subject being treated. The amount of GML in the composition may vary depending on, for example, the nature of the infection or illness; the site of administration; the subject's medical history, subject weight, age, sex, and surface area being treated; and whether the subject is receiving any other medications.

In an embodiment, the topical composition comprises at least one glycol. In one embodiment, the topical composition comprises propylene glycol, polyethylene glycol, or a combination thereof. In one embodiment, the polyethylene glycol has a molecular weight (MW) range from about 300 to about 10,000. In a further embodiment, the polyethylene glycol has a molecular weight of about 300 to about 1,000. In a still further embodiment, the polyethylene glycol has a molecular weight of about 400.

In one embodiment, polyethylene glycol is present in the topical composition. In a further embodiment, the polyethylene glycol has a MW of about 400, about 500 or about 1,000. In one embodiment, the polyethylene glycol is present in the topical composition at a concentration (w/w) of about 15% to about 50%, about 20% to about 40%, or about 25% to about 35%, for example, about 15%, about 20%, about 25%, about 30%, about 35%, about 40%, about 45%, or about 50%. In a further embodiment, both propylene glycol and polyethylene glycol are present in the topical composition. In a further embodiment, propylene glycol is present at a concentration of about 70% to about 80% and polyethylene glycol is present at a concentration of about 20% to about 30%. In even a further embodiment, the polyethylene glycol is polyethylene glycol 400.

In another embodiment, a topical composition comprising GML or a derivative thereof is provided. In a further embodiment, propylene glycol is present in the composition. In yet a further embodiment, propylene glycol is present in the composition at a concentration of about 60% to about 80%, for example, about 60%, about 65%, about 70%, about 71%, about 72%, about 73%, about 74%, about 75%, or about 80%.

In another embodiment, a topical composition comprising GML or a derivative thereof is provided. In one embodiment, the topical composition comprises at least one cellulose derivative. In a further embodiment, the composition comprises one cellulose derivative or two cellulose derivatives. In one embodiment, the cellulose derivative is hydroxypropyl cellulose. In another embodiment, the cellulose derivative is hydroxyethyl cellulose, carboxymethyl cellulose or hydroxymethyl cellulose. In yet another embodiment, the composition comprises a combination of hydroxyethyl cellulose and hydroxypropyl cellulose. In one embodiment, the cellulose derivative is present at a concentration of about 0.1% (w/w) to about 5.0% (w/w). In a further embodiment, multiple cellulose derivatives are present in the composition at the same concentration. In a further embodiment, two cellulose derivatives are present, and each is present at a concentration of about 1.25% (w/w). Cellulose derivatives include, for example, hydroxyethyl cellulose, hydroxypropyl cellulose, methylcellulose, ethyl cellulose, hydroxypropyl methyl cellulose, or cellulose acetate.

In one embodiment, the topical composition comprises GML or a derivative thereof, at least one cellulose derivative, propylene glycol and polyethylene glycol.

In a further embodiment, the composition comprises at least one vegetable oil, for example, at least one of the vegetable oils described above (e.g., palm oil, olive oil, corn oil). In one embodiment, the vegetable oil is present in the composition at a concentration of about 0.1% (w/w) to about 10% (w/w). In a further embodiment, the vegetable oil is present in the composition at a concentration of about 1% (w/w) to about 8% (w/w). In a further embodiment, the vegetable oil is present in the composition at a concentration of about 1% (w/w) to about 6% (w/w). In a further embodiment, the vegetable oil is present in the composition at a concentration of about 1% (w/w) to about 4% (w/w). In one embodiment, the vegetable oil is present in the composition at a concentration of about 0.1% (w/w), about 0.5% (w/w) about 1.0% (w/w), about 1.25% (w/w), about 1.5% (w/w), about 1.75% (w/w), or about 2.0% (w/w).

In one embodiment, the topical composition comprises a vegetable oil and at least one cellulose derivative. For example, in one embodiment, the topical composition comprises hydroxypropyl cellulose and a vegetable oil, or hydroxyethyl cellulose and a vegetable oil, or a combination of hydroxypropyl cellulose, hydroxyethyl cellulose, and a vegetable oil. In one embodiment, the cellulose derivative and the vegetable oil (e.g., palm oil, corn oil, or plant oil), are each present at the same concentration (w/w). In another embodiment, the topical composition comprises petroleum jelly. In a further embodiment, the cellulose derivative and the vegetable oil are each present in the composition at about 1% (w/w) to about 5% (w/w). In a further embodiment, the cellulose derivative is a combination of hydroxypropyl cellulose and hydroxyethyl cellulose, and each is present in the composition at about 1.25% (w/w). In one embodiment, the composition comprises a vegetable oil and two cellulose derivatives. In a further embodiment, the two cellulose derivatives are hydroxypropyl cellulose and hydroxyethyl cellulose, and the total concentration of cellulose derivatives in the composition is about 1.25% (w/w). Cellulose derivatives include, for example, hydroxyethyl cellulose, hydroxypropyl cellulose, methylcellulose, ethyl cellulose, hydroxypropyl methyl cellulose, or cellulose acetate.

In some embodiments, the topical composition comprises one or more accelerants. In a further embodiment, the accelerant is an organic acid, a chelator, an antibacterial agent, an anti-fungal agent, an anti-viral agent, or a combination thereof. In a further embodiment, the accelerant is a chelator. In even a further embodiment, the accelerant is EDTA.

The accelerant, in one embodiment, is EDTA. In a further embodiment, the GML composition comprises EDTA at a concentration of about 0.00005 M, about 0.0005 M, about 0.005 or about 0.05 M. In another embodiment, a chelator is present in the composition at a concentration of about 0.00005 M to about 0.05 M, about 0.0005 M to about 0.005 M, or about 0.005 to about 0.05 M.

In one embodiment, the topical composition comprises both a vegetable oil and an accelerant, for example palm oil and EDTA. In another embodiment, the accelerant is an organic acid and is present in the composition with a vegetable oil. In one embodiment, the topical composition comprises an accelerant and a non-aqueous gel, for example a gel comprising a cellulose derivative. In another embodiment, the topical composition comprises GML or a derivative thereof, a vegetable oil, a non-aqueous gel (e.g., a gel comprising one or more cellulose derivatives) and an accelerant.

In one embodiment, the composition contains at least one pharmaceutically acceptable excipient. Pharmaceutically acceptable excipients are well known to those skilled in the art and may include buffers (e.g., phosphate buffer and citrate buffer), amino acids, alcohols, proteins such as serum albumin, parabens (e.g., methylparaben), or mannitol).

In one embodiment, the pH of the composition is from about 3.5 to about 7.0. In a further embodiment, the pH of the composition is from about 4.0 to about 6.0. In a still further embodiment, the pH of the composition is from about 4.0 to about 4.5.

In one embodiment, the composition comprises GML or a derivative thereof and a pharmaceutically acceptable topical carrier. In one embodiment, the pharmaceutically acceptable topical carrier is a mix of hydrocarbons such as, for example, paraffin wax or petroleum jelly. Petroleum jelly is any water-insoluble, hydrophobic, semi-solid mixture of hydrocarbons. The pharmaceutically acceptable topical carrier can be added to any of the disclosed compositions.

In one embodiment, the composition is a gel. In another embodiment, the composition is a solid, semi-solid, foam, wax, cream, or lotion.

In one aspect, the present invention provides a method of treating a microbial infection in a subject in need thereof. The microbial infection, in one embodiment, is a bacterial, viral, or fungal infection, or a combination thereof.

The disclosed GML topical compositions may be less irritating than currently approved antimicrobial compositions, therefore resulting in a more favorable patient compliance rate, as compared to other antimicrobial compositions presently used in the art.

In one embodiment, the method comprises administering to a subject a topical composition comprising GML or a derivative thereof. In one embodiment, the method comprises topically administering to a subject an effective amount of a composition comprising GML or a derivative thereof, a vegetable oil, and a pharmaceutically acceptable topical carrier. In another embodiment, the method comprises topically administering an effective amount of a composition comprising GML, a non-aqueous gel, and a pharmaceutically acceptable topical carrier. The composition may be given, for example, twice per day for 3-4 days, or 6-7 days. Alternatively, the composition may be given once per day for 7-10 days or 12-14 days.

In one embodiment, the method of treating a microbial infection comprises applying an effective amount of one or more of the GML compositions to at least one skin or mucosal surface of a subject.

In some embodiments, the composition is applied to or impregnated in a wipe, sponge, swab, or other material, and then applied to the skin or mucosal surface of the subject using the respective material. Swab refers to a material suitable for applying a liquid, gel, wax, cream, or lotion to a skin or mucosal surface, or the act of applying a liquid, gel, wax, cream, or lotion to the skin or mucosal surface, or the act of collecting a liquid, gel, wax, cream, lotion, or fluid from the skin or mucosal surface. In some embodiments, the material is attached to a holder, for example a stick, wire, rod, or applicator. In further embodiments, the material attached to a holder is attached at one or both ends thereof. In some embodiments, the wipe, sponge, swab, or other material is pre-loaded or packaged together with the composition.

GML compositions inhibits microbial infection through one or more of several mechanisms that include, for example, direct microbial toxicity; inhibiting entry of the infectious microorganism into the vertebrate cell; inhibiting growth of the microorganism; inhibiting production or activity of virulence factors such as toxins; stabilizing the vertebrate cells; or inhibiting induction of inflammatory or immunostimulatory mediators that otherwise enhance the infectious process.

In one embodiment, direct GML-mediated interruption of bacterial membranes includes interference with the localization of signaling proteins within the membrane, or interference with ligand binding to signaling proteins. In one embodiment, GML has an indirect effect on a two-component signal transduction system and the effect is selected from modifications to membrane structure that interfere with the ability of transmembrane proteins to perform signaling functions; dissipation of the bacterial plasma membrane potential; and alterations of pH gradients across the membranes.

Similar to GML's putative effects on bacterial plasma membranes, GML has been shown to inactivate certain viruses by disrupting viral lipid envelopes.

In some embodiments, the method comprises applying one or more of the compositions to the ears of a subject. For example, in one embodiment, 1 mg/mL GML in a 10% non-aqueous gel is applied to a swab and the swab is rotated around each ear lobe and ear canal 3 times.

In some embodiments, the subject has an ear mite infection caused by, for example, Otodectes cynotis. In some embodiments, the subject has a bacterial infection. Bacterial infections that are treatable with the topical compositions include, but are not limited to, infections caused by the following bacteria: Staphylococci (e.g., S. aureus, S. intermedius, S. epidermidis), Pseudomonas aeruginosa, Proteus vulgaris or mirabilis

Burkholderia cenocepacia, which used to be named Pseudomonas cepacia and is related to Pseudomonas aeruginosa, was killed by GML at concentrations of 500 μg/mL. Mycobacterial species typically produce large amounts of complex fatty acids. However, these organisms were killed by GML at concentrations of ≥50 μg/mL. In addition to inhibiting the growth of gram-positive bacteria, GML inhibited exotoxin production independently from inhibition of growth for all such organisms tested (Staphylococcus aureus, Streptococcus pyogenes, Streptococcus agalactiae, groups C, F, and G streptococci, and Clostridium perfringens). The most susceptible organisms to killing by GML were Peptostreptococcus species, Clostridium perfringens, Bordetella bronchiseptica, and Campylobacter jejuni, all of which were killed by GML (1 μg/mL).

Methods of identifying and diagnosing a bacterial, viral, or fungal infection are generally known by those skilled in the art. To assess whether the disclosed compositions are useful to treat an infection, methods known to those of ordinary skill in the art may be employed.

In one embodiment, a method is provided to remove or kill a biofilm comprising one or more microorganisms. Biofilms can be involved in urinary tract infections, ear infections, and dental diseases such as gingivitis. In one embodiment, the method comprises administering a topical composition by applying it directly to the biofilm. In some embodiments, the methods of the invention comprise administering a second active agent, along with GML or a derivative of GML. The additional active agent may be present in the compositions, or may be administered separately. In one embodiment, the one or more additional active agents prior to, or after, the topical GML composition is administered. For example, the two active agents may be topically administered serially, or administered serially by different routes of administration.

In one embodiment, the additional active agent(s) is administered before, during, or after administration of the composition of the invention. In another embodiment, the additional active agent(s) is administered by the same route as the composition or by a different route. For example, the additional active agent(s), in one embodiment, is administered by one of the following routes of administration such as, for example, topical, intranasal, intradermal, intravenous, intramuscular, oral, vaginal, rectal, otic, ophthalmic, or subcutaneous. The dose of additional active agents depends on, for example, the nature of the infection or illness; the site of administration; subject weight, age, sex, and surface area; concomitant medications; or medical judgment.

Additional active agents include, for example, antibiotics, anti-viral agents, or anti-fungal agents. Antibiotics include, for example, aminoglycosides, carbacephems, cephalosporins, glycopeptides, lincosamides, lipopetides, macrolides, monobactams, nitrofurans, penicillins, polypetides, quinolones, sulfuramides, or tetracyclines. Anti-fungal agents include, for example, those of the azole class, polyene class, or echinocanins class, nucleoside analogues, allylamines, griseofulvin, tolnaftate, or selenium compounds. Anti-viral agents include, for example, acyclovir, ganciclovir, valganciclovir, abacavir, enofovir, lamivudine, emtricitabine, zidovudine, tenofovir, efavirenz, raltegravir, enfuvirdide, maraviroc, ribavirin, amantadine, rimantadine, interferon, oseltamivir, or zanamivir.

### EXAMPLES

The present invention is further illustrated by reference to the following Examples. However, it should be noted that these Examples, like the embodiments described above, are illustrative and are not to be construed as restricting the scope of the invention in any way. As set out below, 5% (50 mg/mL) GML gel-based compositions were used to treat and prevent otitis externa in animals. Additionally, the same GML gel-based compositions could be used to manage inflammatory skin conditions in cats and dogs.

### Example 1

5% w/v GML nonaqueous gel is bactericidal for 54 strains of S. aureus, including highly antibiotic resistant organisms and multiple clonal groups. GML is antimicrobial on contact, killing the organisms in only a few minutes. The estimated chance of S. aureus developing resistance to GML is < 1/10; thus resistance is highly unlikely. 5% GML nonaqueous gel is also a stronger anti-staphylococcal agent than GML alone.

### Example 2

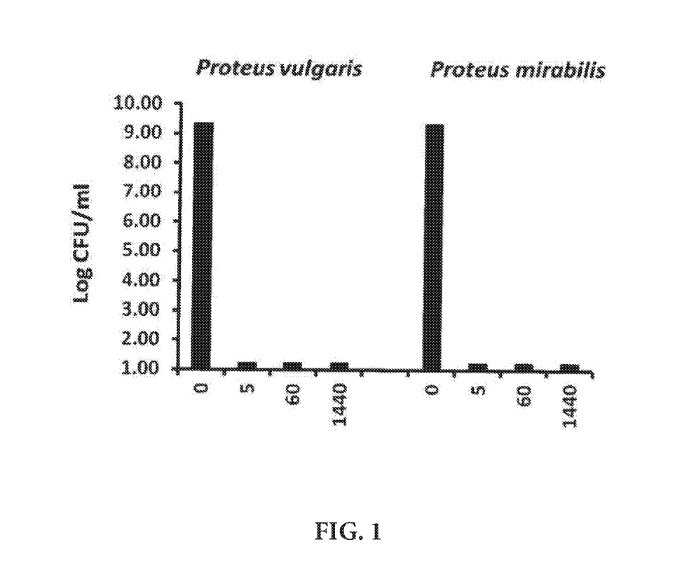

5% GML nonaqueous gel, GML with other accelerants, such as low pH and EDTA, is bactericidal to Pseudomonas species on contact. Bactericidal is defined as a greater than three log reduction in bacterial colony-forming units per milliliter, compared to starting inoculum. Additionally, 5% nonaqueous gel is bactericidal to both Proteus vulgaris and Proteus mirabilis on contact, killing the organisms in less than 5 minutes (see FIG. 1).

### Example 3

Proteus species were added to 0.9 ml of nonaqueous GML gel for the indicated amount of time (1440 minutes=24 hours). Proteus species were added in 0.1 ml volumes after overnight growth in Todd Hewitt media (Difco laboratories, Detroit, Mich.) at 37° C. with 200 revolutions/minute shaking. At selected times, 0.1 ml volumes were removed and plated on Todd Hewitt both containing 1.5% agar plates. These were incubated for 24 hours at 37° C. The experiment was performed in triplicate. In all instances, by 5 minutes post-inoculation no colony-forming units were seen. FIG. 1 provides the results of this study. Differences in colony-forming units/ml compared to 0 time was significant by Student's t test of unpaired normally distributed data at p<<0.001. As a control, spreading 0.1 ml of nonaqueous GML gel onto TH agar plates followed by spreading 0.1 ml volumes of dilutions of Proteus species inocula did not inhibit bacterial growth

The data indicate that nonaqueous 5% GML gel is bactericidal for bacteria that cause ear and skin infections in pets.

### Example 4

In order to assess the effect of GML on biofilms, S. aureus biofilms were grown. S. aureus 128, an organism that expresses toxic shock syndrome toxin-1 (TSST-1), was inoculated at 10⁷/0.1 mL onto the inside of pre-wetted dialysis tubing that had been tied off on one end. A tampon was then inserted into the dialysis tubing and the tubing was immersed under Todd Hewitt broth (Difco Laboratories, Detroit, Mich.) containing 0.8% agar. The open end of the dialysis tubing remained above the agar surface such that the only source of nutrients for the growing microbes was media absorbed across the dialysis tubing.

Tampon sacs were incubated in the solidified agar. At 4, 8, 12, and 16 hours, tampon sacs were removed from the agar, sliced open, and weighed to determine fluid gain. TSST-1 was eluted by addition of phosphate-buffered saline (PBS). The accumulated amount of TSST-1 was quantified by first concentrating the eluted fluids by addition of 4 volumes of absolute ethanol, then resolubilizing in distilled water, and analyzing by Western immunoblot. TSST-1 was not detected in the presence of 5% GML (data not shown).

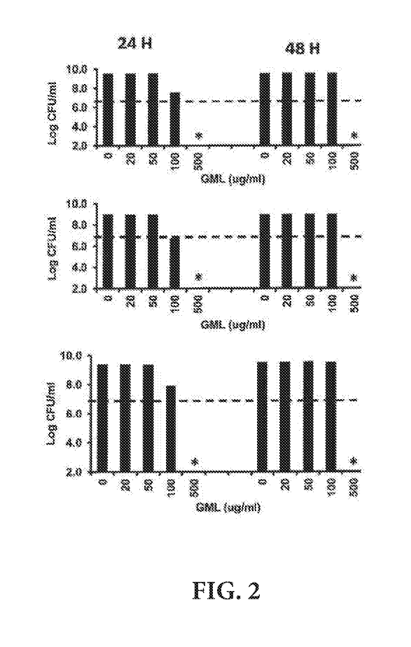

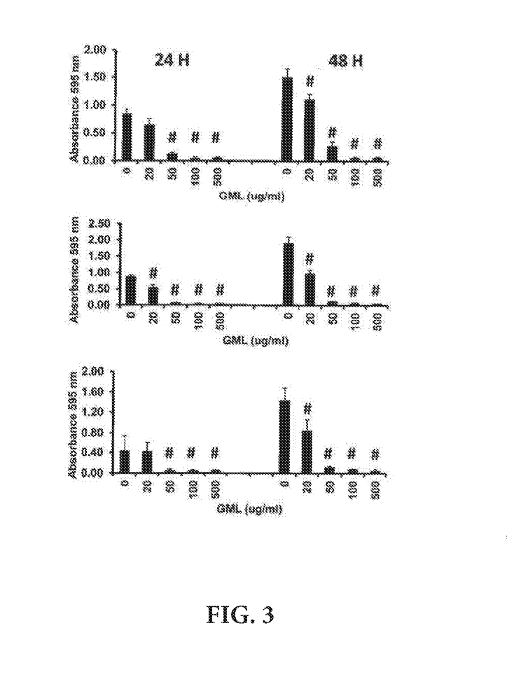

To directly assess the effect of GML on the formation of biofilms, 96 well plastic microtiter plates were inoculated with approximately 10⁶/mL of one of three strains of S. aureus (MN8, a methicillin sensitive strain; MNWH, a methicillin resistant strain; or MW2, a methicillin resistant strain), or with non-typable Haemophilus influenzae. Wells were cultured stationary at 37° C. for 24 and 48 hours (see, e.g., FIGS. 2 and 3). As a control, in one set of three wells for each microbe, the wells were agitated 3 times by pipetting up and down. The bactericidal activity of GML was determined by measuring CFU/mL in supernatants. After removal of supernatants, wells were washed three times with PBS to remove unbound cells, and were then treated with crystal violet for 30 minutes. Wells were again washed three times with PBS to remove unbound crystal violet. Finally, wells were treated with ethanol to solubilize biofilm associated crystal violet. Absorbance at 595 nm was determined by an ELISA reader to measure biofilm formation.

FIGS. 2 and 3 provide the results of the study. Growth of all three S. aureus strains was completely inhibited by GML at 500 μg/mL at both 24 and 48 hours, as measured by CFU/mL (FIG. 2; dashed line indicates starting inoculum size; *significant reduction in mean CFU/mL compared to starting inoculum, p<0.001.). In contrast, at 10 fold lower GML concentrations than necessary to inhibit bacterial growth, biofilm formation was significantly inhibited as measured by reduced crystal violet staining of retained biofilm material in wells of the microtiter plates (FIG. 3; significant reduction in mean absorbance at 595 nm compared to no GML wells, p<0.01).

### Example 5

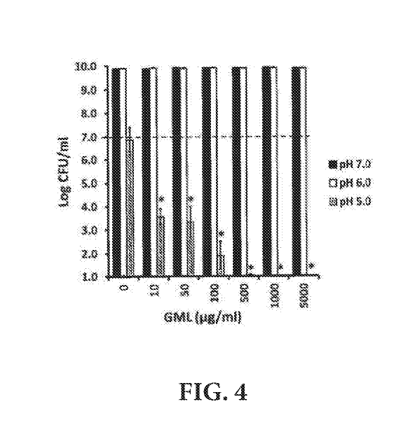

The effect of GML at a range of concentrations and in the presence of a range of pH levels on the growth of Pseudomonas aeruginosa was determined. P. aeruginosa (strain PA01) was inoculated in Todd Hewitt broth at 5.7×106/mL. GML was added to cultures at a range of concentrations from 10 μg/mL to 5000 μg/mL, and the pH was adjusted to 5.0, 6.0, or 7.0. CFU/mL was determined after 24 hours of incubation. FIG. 4 provides the results of this study. At a pH of 6.0 or 7.0, no concentration of GML was inhibitory for the growth of P. aeruginosa. A pH of 5.0 in the absence of GML was somewhat inhibitory for the growth of P. aeruginosa. However, the addition of GML to the cultures at a pH of 5.0 further inhibited P. aeruginosa growth in a dose-dependent manner (*p<0.001; dashed line indicates starting inoculum size).

### Example 6

The plant spider mite Tetranychus urticae, an arachnid that causes infections of plants, including tomatoes, peppers, potatoes, beans, corn, strawberries, cannabis, and house plants, such as Schefflera actinophylla was tested. These spider mites live on the underside of leaves and cause damage by puncturing plant cells. They also spin silk webs and quickly become resistant to pesticides. Schefflera plants can be ridded of spider mites by washing the leaves with aqueous GML at 500 μg/mL in two administrations. This data indicates that GML is cidal to spider mites and likely to be cidal to other mites.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method of treatment or prophylaxis, the method comprising:
(a) identifying a subject likely to have been, or likely to be, exposed to an infectious microorganism or mite, wherein the mite or microorganism is of a genus selected from the group consisting of Otodectes, Staphylococcus, Pseudomonas, and Proteus; and
(b) administering to the subject a glycerol monolaurate (GML) composition that
(i) kills, or inhibits the growth of, the infectious microorganism or mite, and
(ii) comprises a compound selected from the group consisting of: 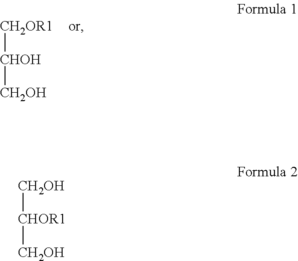
wherein R1 is: CO(CH₂)₁₀CH₃.

2. (canceled)

3. The method of claim 1, wherein GML is present in an amount of 10-100 mg/mL.

4. The method of claim 1, wherein GML is present in an amount of 30-70 mg/mL.

5. The method of claim 1 further comprising a vegetable oil.

6. The method of claim 1 further comprising a cellulose derivative.

7. The method of claim 1 further comprising one or more accelerants.

8. The method of claim 1 further comprising an additional active ingredient.

9. The method of claim 1 further comprising an additional active ingredient selected from an anti-biotic, anti-viral, anti-fungal, or combination thereof.

10. The method of claim 1, wherein the gel-based composition is glycerol, vegetable oil, petroleum jelly, or combination thereof.

11. The method of claim 1, wherein the glycerol-based compound is a gel, cream, or foam.

12. A composition comprising an effective amount of glycerol monolaurate (GML) or a derivative thereof, in a gel-based composition that kills, or inhibits the growth of, an infectious microorganism or mite in a subject likely to have been, or likely to be, exposed to the infectious microorganism or mite, wherein the mite or microorganism is of a genus selected from the group consisting of Otodectes, Staphylococcus, Pseudomonas, and Proteus.

13. The composition of claim 12, wherein GML is present in an amount of 10-100 mg/mL.

14. The composition of claim 12, wherein GML is present in an amount of 30-70 mg/mL.

15. The composition of claim 12 further comprising a vegetable oil.

16. The composition of claim 15, wherein vegetable oil is present in an amount of 0.1% (w/w) to 10% (w/w).

17. The composition of claim 12, further comprising a cellulose derivative.

18. The composition of claim 12 further comprising one or more accelerants.

19. The composition of claim 12 further comprising an additional active ingredient.

21-23. (canceled)

24. The composition of claim 12 further comprising an additional active ingredient selected from an anti-biotic, anti-viral, anti-fungal, or combination thereof.
